(19) United States
(12) Patent Application Publication
(10) Pub. No.: US 2005/0137175 A1
(43) Pub. Date: Jun. 23, 2005

(54) Use of ecdysteroids for preparing dermatological or cosmetological anti-hair loss compositions

(75) Inventors: Bruno Bernard, Neuilly-sur-Seine (FR); Brigitte Gautier, Les Ulis (FR)

Correspondence Address: LERNER, DAVID, LITTENBERG,;KRUMHOLZ & MENTLIK, 600 SOUTH AVENUE WEST, WESTFIELD, NJ, 07090, US

(73) Assignee: L'OREAL, Paris (FR)

(21) Appl. No.: 11/017,618

(22) Filed: Dec. 20, 2004

Related U.S. Application Data
(60) Provisional application No. 60/575,441, filed on May 28, 2004.

(30) Foreign Application Priority Data
Dec. 22, 2003 (FR) 03 15157

(51) Int. Cl. A61K 31/56

(52) U.S. Cl. 514/169

(57) ABSTRACT

The present invention relates to the use of at least one ecdysteroid for preparing cosmetic or dermatological compositions intended to prevent hair loss.

### CROSS-REFERENCE TO RELATED APPLICATIONS

The present invention claims the benefit of filing date of U.S. Provisional Application No. 60/575,441, filed May 28, 2004, the disclosure of which is hereby incorporated herein by reference.

The present invention relates to the use of at least one ecdysteroid for preparing cosmetic or dermatological compositions intended to prevent hair loss.

Ecdysteroids are a group of 2,3,14-trihydroxy-Δ-7-6-ketosteroids. These molecules are well known in the literature and are cited in the Merck Index, 10th edition, 1983, page 505, No. 3470.

It is known that ecdysteroids, and in particular ecdysterone (or β-ecdysone) play an important role both in the animal kingdom in insects, where ecdysterone is involved in particular in metamorphosis, and in the plant kingdom, where these substances appear to play a role in flowering.

It has recently been discovered that ecdysteroids regulate epidermal keratinocyte differentiation. In the hair follicle, these hormones regulate, or even increase, the synthesis of keratin, which is the main constituent of the pilar shaft (U.S. Pat. No. 5,609,873). Those skilled in the art may refer to the article by Eckert and Rorke (1989, Environment Health Perspective, 80: 109-116).

Ecdysterone, in particular, has been described as an agent for improving the appearance of the skin (CN 86 1 06791A) and of the hair (U.S. Pat. No. 5,609,873), and also the constitutive quality of the hair fibre (FR 93 10138). In addition, ecdysterone is known to stimulate hair regrowth in the C3H murine model (H. Ishida et al., 1999, Biol. Pharma. Bull. 22: 1189-1192).

Ecdysterone, an insect-moulting hormone, is known to modify the expression of a certain number of genes in insects via a specific nuclear receptor. Given the structural similarity between ecdysterone and steroids such as androgens, oestrogens, etc, which are known to modify hair growth, the applicant has had the idea of evaluating the effect of ecdysterone on hair follicle growth and survival.

The applicant has thus discovered, surprisingly and unexpectedly, that ecdysterone is capable of promoting human follicle survival, in vitro and of maintaining, without stimulating, firstly, the mitotic activity of the cells of the human hair follicle matrix and, secondly, the programmes for differentiation of the outer sheath and of the inner sheath of the follicle, while at the same time ensuring the integrity of the pilar shaft.

A subject of the invention is thus the use of ecdysteroids in the preparation of a composition for cosmetic or dermatological use.

A subject of the invention is also a cosmetic treatment process comprising the use of this composition for preventing hair loss.

Other subjects of the present application will emerge on reading the description and the examples which follow.

A subject of the present invention is thus the use of at least one ecdysteroid or of at least one ecdysteroid analogue or of at least one ecdysteroid derivative, for preparing a cosmetic or dermatological composition intended to prevent hair loss.

The term “ecdysteroid analogue” is intended to mean steroids that are generally characterized by a cis-junction of the A and B rings, a 7-en-6-one chromophore, and a 14α-hydroxy group. These analogues generally have a backbone of the cholesterol (C27) or phytosterol (C28, C29) type (C. Blais et al, 1996, Biochem. J. 320: 413-419). Currently, more than 250 structural analogues have been identified (Laffont R. and Wilson I D, 1996, The ecdysone handbook, 2nd edition, The Chromatographic Society, Nottingham, U.K., pp 525; see internet site http://ecdybase.org).

Ecdysteroid derivatives may be products from oxidation of ecdysteroids or of these analogues, from reduction or from conjugation. Preferably, the ecdysteroid derivatives are acyl, hydroxyl or deoxyl derivatives.

Preferably, the ecdysteroids, ecdysteroid analogues or ecdysteroid derivatives (collectively referred to as “at least one ecdysteroid compound,” as the context permits) chosen from ecdysterone, ecdysone, muristerone A, ponasterones A, B and C, inokosterone, phytoecdysteroids such as limnantheoside A and limnantheoside B (J. Preston-Mafham & L. Dinan, 2002, Z. Naturforsch. 57c: 144-152), ajugasterone B and ajugasterone C, turkesterone, podecdysone, stachysterone, or 7,9(11)-diene-6-one ecdysteroids such as kaladasterone, dacryhainansterone or 25-hydroxydacryhainansterone (P. C. Bourne et al., 2002, J. Insect Sci. 2: 11).

More preferably, the ecdysteroid in question is ecdysterone or an ecdysterone analogue.

The ecdysteroids, their analogues or their derivatives are obtained in isolated form, or in the form of an extract from any of the available natural sources, or else by means of a process of chemical synthesis. The main natural sources of ecdysteroids are insects, marine invertebrates and a large number of plants. The ecdysteroids can thus be extracted from Achyranthes bidentata, from Achyranthes aspera, from various species of Paris, from Polypodium vulgare, from Cyanotis arachnoidea, from Ajuga decumbens, from Ajuga iva, from Pfaffia paniculata, from Pfaffia iresinoides, from Vitex glabrata, from Sesuvium portulacastrum, from Serratula sogdiana, from Serratula tinctoria, from Rhaponticum integrifolium, from Rhaponticum carthamoides, from various species of Silene, from Lychnis flos-cuculi, from Cyathula officinalis, from Cyathula capitata or from Bombyx mori. More particularly, among the plants which can be used for extraction of the ecdysteroids, in particular of ecdysterone, mention may be made of Serratula sogdiana and Rhaponticum integrifolium, which have the advantage that they can be cultivated.

Moreover, a certain number of ecdysteroids are commercially available. Ecdysterone is in particular available from Sigma-Aldrich under the reference H5142.

The weight-amount of ecdysteroid, of ecdysteroid analogue or of ecdysteroid derivative is between 0.001 and 10% relative to the total weight of the composition.

Preferably, the weight-amount of ecdysteroid, of ecdysteroid analogue or of ecdysteroid derivative is between 0.01 and 5% relative to the total weight of the composition.

The cosmetic composition according to the invention may also comprise other cosmetic agents intended to promote hair growth, such as for example aminexil (2,4-DPO, 2,4-diaminopyrimidine-N-oxide).

The composition according to the invention may also comprise water and/or one or more cosmetically acceptable organic solvents. The solvents may be chosen from the group consisting of hydrophilic organic solvents, lipophilic organic solvents, amphiphilic solvents or mixtures thereof.

Among the hydrophilic organic solvents, mention may be made, for example, of linear or branched lower monoalcohols having from 1 to 8 carbon atoms, such as ethanol, propanol, butanol, isopropanol, isobutanol, optionally oxyethylenated polyethylene glycols, polyols such as propylene glycol, isoprene glycol, butylene glycol, glycerol, sorbitol and its derivatives, glycol ethers and propylene glycol ethers.

As amphiphilic organic solvents, mention may be made of polyols such as propylene glycol derivatives.

As lipophilic organic solvents, mention may, for example, be made of fatty esters.

The composition according to the invention may also comprise adjuvants such as fatty substances, preserving agents, stabilizers, opacifiers, softeners, silicones, foaming agents, antioxidants, pH regulators, emulsifying agents, conventional hydrophilic or lipophilic thickeners and/or gelling agents, hydrophilic or lipophilic active agents, fragrances, emulsifiers, sequestering agents, polymers, acidifying or basifying agents, fillers, free-radical scavengers, ceramides, moisturizers, vitamins, surfactants, anti-dandruff agents, propellants, dyes, or any other adjuvant normally used in cosmetics.

The amount of these various adjuvants are those conventionally used in the field in question. Those skilled in the art will take care to choose the optional compound(s) to be added to the composition according to the invention so as not to impair, or not substantially impair, the advantageous properties attached to the composition in accordance with the invention.

The compositions in accordance with the invention are particularly suitable in cosmetic and/or dermatological terms, and do not cause any irritation of the scalp, even after prolonged contact without rinsing.

The composition according to the invention may be in any of the pharmaceutical forms normally used for cosmetic or dermatological topical application, in particular in the form of an aqueous, aqueous-alcoholic or oily solution, of an oil-in-water or water-in-oil or multiple emulsion, or of an aqueous or oily gel.

The composition may have the appearance of a milk, of a gel, of a mousse, of a serum or of a lotion.

The compositions defined above can be applied to the hair or the scalp, and can be applied, for example, after washing the scalp.

These compositions are intended to be used in cases of hair loss, whether this loss is of natural origin or of drug-related origin, i.e. subsequent to the absorption of medicinal products for which hair loss is a side effect.

A subject of the present invention is also a process of cosmetic treatment of the scalp with the aim of preventing hair loss, this process consisting in applying, to the areas in question, a cosmetically effective amount of a composition containing at least one ecdysteroid, an ecdysteroid analogue or an ecdysteroid derivative.

The following examples are intended to illustrate the invention without, however, being limiting in nature.

### EXAMPLE 1

### Effect of Ecdysterone on Human Hair Follicle Survival in Vitro

We had developed and patented a model for a surviving human follicle in vitro (FR 95-08465) making it possible to evaluate the effect of a substance on follicle survival and extension.

We used this in vitro model for evaluating the effect of ecdysterone on hair follicle growth and survival. Various concentrations of this substance were tested: 0.01 μg/ml, 0.1 μg/ml, 1 μg/ml and 10 μg/ml.

The statistical tests performed on the entire sample show that the differences observed between the groups are significant.

The statistical analysis thus shows that ecdysterone has a positive effect on human hair follicle survival in vitro. Specifically, ecdysterone at the concentrations from 0.01 μg/ml, 0.1 μg/ml and 10 μg/ml significantly increases human hair follicle survival under these culture conditions.

### EXAMPLE 2

### Effects of Ecdysterone on the Maintenance of Hair Follicle Integrity in Vitro

In order to characterize more fully the effect of this substance on the hair follicle, we monitored the mitotic activity of the bulb cells and also the various specific differentiation programmes of the outer sheath, of the inner sheath and of the pilar shaft, in the presence or absence of this substance.

The integrity of the follicle compartments after 14 days of culture was thus monitored by means of antibodies that recognize markers specific for each of these compartments: the mitosis marker Ki67, specific for the matrix (S. Commo & B. A. Bernard, 1997, Br. J. Dermatol. 137: 31-38); keratin K14, specific for the outer sheath (ibid.); transglutaminase, specific for the inner sheath (ibid.); and keratin hHa5, specific for the pillar shaft, and in particular the keratogenic zone and the cuticle (L. Langbein et al., 1999, J. Biol. Chem. 274: 19874-19884).

After 14 days of culture, in the absence of ecdysterone, histological analysis of the hair follicle shows that the Ki67 labelling decreases, keratin K14 is ectopically expressed, the transglutaminase expression points to a thickening and a disorganisation of the inner sheath, and keratin hHa5 shows an alteration in the shaft cuticle. On the other hand, after 14 days of culture in the presence of ecdysterone at 0.01 μg/ml, all the markers studied conserve an expression that is comparable to that of the reference follicle at D=0.

Ecdysterone therefore opposes the spontaneous degradation of the follicle and maintains the integrity of the follicular compartments after 14 days of culture in vitro.

### EXAMPLE 3

### Cosmetic Composition No. 1 Intended to Prevent Hair Loss

The following components are weighed out, mixed and solubilized at ambient temperature.

| | |
|---|---|
| Ecdysterone | 9.0 |
| Polypropylene glycol | 5.0 |
| Vitamin E | 0.1 |
| Diphenhydramine hydrochloride | 0.1 |
| Dipotassium glycyrrhetinate | 0.1 |
| Methylparabene | 0.2 |
| Ethanol | 50.0 |
| Fragrance | 1.0 |

### EXAMPLE 4

### Cosmetic Composition No. 2 Intended to Prevent Hair Loss

The following components are weighed out, heated separately and dissolved at 80° C. before mixing.

| | |
|---|---|
| Ponasterone C | 0.5 |
| Inokosterone | 0.5 |
| Stearyl methylammonium chloride | 3.5 |
| Polypropylene glycol | 5.0 |
| Liquid paraffin | 2.0 |
| Methylpolysiloxane | 2.0 |
| Behenyl alcohol | 4.0 |
| Fragrance | 0.5 |
| Purified water | 82.0 |

1. A method of preventing hair loss comprising:
providing a composition comprising at least one ecdysteroid compound in a cosmetically effective amount and topically applying same to the scalp or hair so as to prevent hair loss.

2. The method of claim 1, wherein said at least one ecdysteroid compound is an ecdysteroid, and ecdysteroid analogue or ecdysteroid derivative.

3. The method of claim 2 wherein said ecdysteroid, ecdysteroid analogue or ecdysteroid derivative is chosen from ecdysterone, ecdysone, muristerone A, ponasterones A, B and C, inokosterone, limnantheoside A and limnantheoside B, ajugasterone B and ajugasterone C, turkesterone, dacryhainansterone, kaladasterone, podecdysone, stachysterone, or the 7,9(11)-dien-6-one ecdysteroids.

4. The method of claim 2, wherein said at least one ecdysteroid compound is an ecdysteroid

5. The method of claim 4 wherein said ecdysteroid is ecdysterone.

6. The method of claim 2 wherein said at least one ecdysteroid compound is an ecdysterone analogue.

7. The method of claim 2 wherein said at least one ecdysteroid compound is an ecdysterone derivative.

8. The method of claim 1 wherein said cosmetically effective amount of said at least one ecdysteroid compound is between 0.001 and 10% relative to the total weight of said composition.

9. The method of claim 8 wherein said cosmetically effective amount of said at least one ecdysteroid compound is between 0.01 and 5% relative to the total weight of said composition.

10. The method of claim 1 wherein said composition further comprises a cosmetic agent.

11. The method of claim 1 wherein said cosmetic agent promotes hair growth.

12. The method of claim 11 wherein said cosmetic agent 2,4-DPO.

13. The method according to any one of claims 1 or 11, further comprising water, alcohol or one or more cosmetically acceptable organic solvents.

14. The method according to any one of claims 1 or 11, further comprising one or more cosmetically acceptable adjuvants.

15. The method according to any one of claims 1 or 11, wherein said composition is in the form of an aqueous or aqueous-alcoholic solution, or an aqueous or aqueous-alcoholic gel.

16. The method according to any one of claims 1 or 11, wherein said composition is in the form of a milk, of a gel, of a mousse, of a serum or of a lotion.

17. A method of promoting human follicle survival comprising: providing a composition comprising at least one ecdysteroid compound in a cosmetically effective amount and topically applying same to the scalp or hair so as to prevent hair loss.

18. The method of claim 17, wherein said at least one ecdysteroid compound is an ecdysteroid, and ecdysteroid analogue or ecdysteroid derivative.

19. The method of claim 18 wherein said ecdysteroid, ecdysteroid analogue or ecdysteroid derivative is chosen from ecdysterone, ecdysone, muristerone A, ponasterones A, B and C, inokosterone, limnantheoside A and limnantheoside B, ajugasterone B and ajugasterone C, turkesterone, dacryhainansterone, kaladasterone, podecdysone, stachysterone, or the 7,9(11)-dien-6-one ecdysteroids.

20. The method of claim 18, wherein said at least one ecdysteroid compound is an ecdysteroid

21. The method of claim 20 wherein said ecdysteroid is ecdysterone.

22. The method of claim 17 wherein said cosmetically effective amount of said at least one ecdysteroid compound is between 0.001 and 10% relative to the total weight of said composition.

23. The method of claim 22 wherein said cosmetically effective amount of said at least one ecdysteroid compound is between 0.01 and 5% relative to the total weight of said composition.

24. The method of claim 22 wherein said composition further comprises a cosmetic agent.

25. The method of claim 24 wherein said cosmetic agent 2,4-DPO.

26. The method according to any one of claims 17, 22 or 25, further comprising water, alcohol, a cosmetically acceptable organic solvent or a cosmetically acceptable adjuvant.

27. The method according to claim 26, wherein said composition is in the form of an aqueous or aqueous-alcoholic solution, or an aqueous or aqueous-alcoholic gel.

28. The method according to claim 26, wherein said composition is in the form of a milk, of a gel, of a mousse, of a serum or of a lotion.

29. A composition for preventing hair loss comprising: at least one ecdysteroid compound selected from ecdysterone, ecdysone, muristerone A, ponasterones A, B and C, inokosterone, limnantheoside A and limnantheoside B, ajugasterone B and ajugasterone C, turkesterone, dacryhainansterone, kaladasterone, podecdysone, stachysterone, or the 7,9(11)-dien-6-one ecdysteroidsin in a cosmetically effective amount and a cosmetic agent, water, alcohol, a cosmetically acceptable organic solvent or a cosmetically acceptable adjuvant.

30. The composition of claim 1 wherein said cosmetically effective amount of said at least one ecdysteroid compound is between 0.001 and 10% relative to the total weight of said composition.
